(19) United States
(12) Patent Application Publication
(10) Pub. No.: US 2020/0057063 A1
(43) Pub. Date: Feb. 20, 2020

(54) METHOD FOR DETECTING PRESENCE OF A THIOETHER AND KIT FOR DETECTING SAME

(71) Applicant: Kimberly-Clark Worldwide, Inc., Neenah, WI (US)

(72) Inventors: John Gavin MacDonald, Decatur, GA (US); Stephen Quirk, Alpharetta, GA (US); Jose K. Abraham, Neenah, WI (US); Priyadarshini Gadgil, Roswell, GA (US)

(21) Appl. No.: 16/608,153

(22) PCT Filed: Apr. 24, 2017
(86) PCT No.: PCT/US17/29176

§ 371 Date: Oct. 24, 2019

(51) Int. Cl. G01N 33/569

(52) U.S. Cl. CPC G01N 33/56905; G01N 2333/445

(57) ABSTRACT

A method for detecting a malaria infection whereby providing a mouth cleansing product to a user then asking the user to blow or breathe onto or into a capture mechanism, wherein the capture mechanism may contain a thioether detection mechanism such as Michler's Hydrol; and analyzing whether a thioether detection mechanism displays any loss of the thioether detection mechanism. The invention also discloses a kit for indicating the presence of malaria by instructing the user to exhale through a capture mechanism after using a mouth cleansing product, wherein the kit includes a thioether detection mechanism such as Michler's Hydrol.

### TECHNICAL FIELD

The invention provides for a method and kit for detecting the presence of thioethers in a user's breath. The invention specifically provides for methods and kits for instructing a user to use a mouth cleansing product, and thereafter causing the user to blow or breathe onto or into a substrate, strip, straw, tube, or a similar device thereof wherein the device comprises a thioether detection mechanism. Once the user breathes or blows into or onto the thioether detection mechanism, the user's breath is analyzed with a thioether detection mechanism to determine if a thioether is present in a user. The thioether detection mechanism may be 4,4′-bis(dimethylamino)-benzhydrol (BDMB or “Michler's Hydro”) or a similar compound thereof.

### BACKGROUND OF THE DISCLOSURE

The present invention relates to a method and kit for detecting thioethers in a user's breath and uses thereof, in particular for diagnosing malaria.

Globally an estimated 3.2 billion people in 97 countries are at risk of malaria infection, (Plasmodium falciparum), and in 2013, an estimated 198 million cases and 584,000 deaths were attributed to this infection.

Currently, malaria diagnosis remains mostly based on a method using powerful microscopes to look for parasites in blood. This method's accuracy depends on the skill of the operator and the use of well-maintained equipment, and low levels of parasitemia can be challenging to detect.

Rapid accurate diagnosis of malaria is important to provide adequate treatment, conserve valuable drugs, and help prevent the emergence of resistant strains of malaria parasites. There is therefore a clear need for an affordable, portable and rapid indicator for diagnosing malaria.

Human breath offers an attractive alternative as the basis for simple, noninvasive diagnosis of malaria.

Accordingly, as the world starts to work toward elimination of malaria, there is a need for a sensitive, simple, inexpensive, reliable and convenient diagnostic test to detect early and hidden cases that can be performed essentially anywhere in the world from the comfort of a home to a village in a remote area.

### SUMMARY OF THE DISCLOSURE

The invention describes a method for rapidly diagnosing malaria in a human or animal user (‘user’). The invention provides for methods and kits for instructing a user to use a mouth cleansing product, and thereafter causing the user to blow or breathe onto or into a capture mechanism such as a substrate, straw, vial, tube or a similar device thereof wherein the capture mechanism can include a thioether detection mechanism. Once the user breathes or blows into or onto the thioether detection mechanism, the user's breath is analyzed to determine if a thioether is present in a user.

The thioether detection mechanism is very sensitive to low levels (10 parts per billion (ppb) and more preferably 20 ppb) of sulfide and/or thioether containing odors. On exposure to low levels of these odors the thioether detection mechanism color intensity discharges from a breath testing device thereby indicating the presence of a thioether for rapidly diagnosing malaria to the user.

Suitable thioether detection mechanisms are 4,4′-bis(dimethylamino)-benzhydrol (Michler's hydrol or BDMB) and related dyes. Michler's Hydrol was identified and demonstrated to be sensitive to very low levels (about 15 ppb or above) of sulfur and/or amine volatile compounds.

Simple capture mechanisms such as breath testing devices were also developed, wherein a thioether detection mechanism is discharged when the user blows or breathes into or onto the capture mechanism thus indicating the presence of a thioether in the breath of the user thus rapidly diagnosing malaria to the user.

The capture mechanism may include a simple carrier portion defining a passage, such as a tube, straw, vial or a similar device thereof wherein the devices are transparent, semi-transparent, or a combination thereof. The capture mechanism may also contain the thioether detection mechanism inside and outside of it and may be open on at least one end. While the invention will be described hereinafter with particular reference to straws and tubes, it will be understood that the invention is useful with various other shapes as well. For example, the shape of the passageway may be cylindrical, triangular, square, almond-shaped and so forth.

Capture mechanisms disclosed herein can alternately include a simple bag, vial or balloon or any similar device thereof. Or the capture mechanisms can later be transferred to a thioether detection mechanism as required. For example, attaching a capture mechanism to an analytical chemical instrumentation such as gas chromatography mass spectrometry, liquid chromatography mass spectrometry.

The thioether detection mechanism such as Michler's Hydrol may be in the form of a powder, in solution, or may be coated onto a substrate, such as a cellulose tissue or paper, a non-woven polypropylene/polyethylene substrate, a woven substrate, glass fiber, cotton, silk, rayon and so forth. The straw may be flattened to allow for easier storage, while still permitting a user to blow through the passage in the tube.

In one embodiment, the thioether detection mechanism includes a conveniently sized (about 0.5 inches or larger) and discreet plastic tube, such as a drinking straw containing the thioether detection mechanism. A more preferred embodiment may include a plastic tube of about 2 inches or larger. The thioether detection mechanism can be applied to a strip which is inserted into the straw and can be used in powder form or can be incorporated into a solution inside the straw.

In order to test a user's breath, the user first uses a mouth cleansing product from about 5 seconds to 20 seconds, discards it and then breathes or blows into or onto the device, and the thioether detection mechanism discharges minimally. A minimal discharge is calculated as a change in color that is still visible to the human eye as measured as ΔE* of 3 units or greater or by using a UV/VIS spectrophotometer where the Absorption value is equal or greater than A=0.1 when measured at a wavelength of A=605 nm). This capture mechanism device therefore rapidly detects malaria in the user and allows him or her to take action (48 hours or less after exposure to the malaria parasite) before traditional detection, i.e. minimum of four days after exposure to the malaria parasite.

A kit for containing and dispensing the devices were also developed. In one embodiment, the kit containing the devices and a separate mouth cleansing product are designed so that the devices and/or mouth cleansing product can be sold together or separately from one another.

### BRIEF DESCRIPTION OF DRAWINGS

FIG. 1 shows a simple unassembled capture mechanism such as breath testing devices according to one embodiment of the invention where the picture on the left includes a thioether detection mechanism on a substrate prior to using and where the picture on the right shows a discharge of thioether detection mechanism after using the capture mechanism.

FIG. 2 shows a simple bottle of mouth cleansing product.

FIG. 3 shows a LED meter which reads and measures blue dye (wavelength max.=605 nm) that is used in Michler's Hydrol.

FIG. 4 shows the capture mechanism with an optionally removable bag attached on one end of the capture mechanism;

FIG. 5 shows a further embodiment of the capture mechanism according to the invention.

FIG. 6 shows an additional embodiment of the capture mechanism according to the invention.

Repeat use of reference characters in the present specification and drawings is intended to represent the same or analogous features or elements of the disclosure.

### DETAILED DESCRIPTION OF THE DISCLOSURE

The invention provides simple capture mechanism devices (A) which are able to detect levels as low as about 10 parts per billion (ppb) of sulfides and/or thioether compounds in a user's breath. The capture mechanism devices include a visual indicating agent. The visual indicating agent disclosed herein is a thioether detection mechanism (D). The capture mechanism device is portable, discreet, disposable and relatively inexpensive to produce.

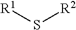

A thioether is a functional group in organosulfur chemistry with a connectivity as disclosed above.

In one embodiment, the thioether detection mechanism is 4,4′-bis(dimethylamino)-benzhydrol also known as BDMD, Michler's Hydrol or MH (‘Michler's Hydrol’ hereinafter). Michler's Hydrol is a blue color dye found at a wavelength of 650 nanometers (nm) and is sensitive to sulfur-containing odors. The blue color is very sensitive to thioethers causing a decrease or discharge of the blue color from the capture mechanism after the user blows into the device. Sulfur odors including thioethers can be produced in the breath not only from medical conditions such as malaria, but also can be present due to bacteria and plaque in the mouth that can form unpleasant smell in the breath. Since Michler's Hydrol is known to be sensitive to the detection of sulfur odors, a mouth washing rinse (FIG. 2) has been shown to effectively remove bad breath and the bacteria for a short period of time by reacting with the plaque on the tongue and the teeth and gum areas in the mouth.

More specifically, the mouth cleansing product should be kept in the user's mouth preferably for about 10 seconds to remove bad breath and bacteria. The time though for keeping the mouth washing rinse in a user's mouth is dependent upon the concentration of the anti-bacteria ingredient and the vigor a user uses to swish around the mouth cleansing product. Accordingly, one embodiment is to keep the mouth cleansing product in a user's mouth for about one second, another embodiment is for about 5 seconds, a further embodiment is for about 10 seconds, an additional embodiment is for about 15 seconds, and yet another embodiment is for about 20 seconds and a final embodiment for cleansing is for more than 20 seconds.

The blowing or breathing onto or into the capture mechanism should be preferably administered in about 5 minutes or less after using the mouth washing rinse. And a mouth cleansing product that contains bacteria fighting ingredients is preferred. An additional embodiment of time for a user to breath or blow onto or into the capture mechanism after using the mouth washing rinse is about 30 seconds or less, or 1 minute or less, or three minutes or less, or 10 minutes or less, or 20 minutes or less, or even more than 20 minutes.

In a further embodiment, the amount of blowing or breathing a user provides onto or into the capture mechanism can be one full breath of air, a short quick breath, multiple breathes of air or an amount of air the user breathes or blows that visibly shows any loss of the thioether detection composition.

Another embodiment of the invention depicts utilizing an appropriate mouth cleansing product to temporarily remove and/or dilute a bad breath (typically hydrogen sulfide and methyl mercaptan) in order that it does not interfere with a thioether such as Michler's hydrol and give a false positive. Common commercially available name branded mouth washes that can be used are Listerine®, Crest®, ACT®, Biotene®, Rembrandt®, Jason®, BreathRX®, CloSYS® or any other similar commercially available mouth rinse on the market for sale. Alternatively, teeth brushing or rinsing with water, or rinsing with baking soda solution, or rinsing with an alcohol solution are appropriate mouth cleansing products to temporarily remove bad breath from a user.

A mouth cleansing product as defined and used herein is any of the aforementioned common commercially available name branded mouth washes, non-name branded mouth washes as well as the utilization of teeth brushing with any commercially available toothpaste, water, baking soda solution, or rinsing with an alcohol solution (of any concentration thereof) whereby temporarily removing bad breath from a user.

Furthermore a mouth cleansing product as disclosed herein is defined as a mouthwash, mouth rinse, oral rinse or mouth bath, wherein it is a liquid which is held in the mouth passively or swilled around the mouth by contraction of the perioral muscles and/or movement of the head, and may be gargled, where the head is tilted back and the liquid bubbled at the back of the mouth. By instructing a user to administer a mouth cleansing product prior to utilizing a capture mechanism such as a breath testing device, a reduction in the false detection of malaria based on thioethers that are present in a user's breath due to plaque and bacteria in a user's mouth can be obtained. In other words, utilizing a mouth cleansing product can increase the accuracy of detecting a disease in a user that produces thioethers, such as malaria.

A substrate (B), typically a cellulose tissue, may also be coated with nanoparticles to provide high surface area coating on the substrate, i.e. higher than the cellulose fiber by itself. The cellulose tissue may be given a boost in surface area by coating it with nanoparticles. The treated substrate may then be coated with a thioether detection mechanism dye. It is believed that this high surface area coating helps spread the dye over the silica surface to provide a coating with higher surface area and thus improve the sensitivity of the capture mechanism. The nanoparticles used on the coating of the surface area is less than 100 nanometers to about 1 nanometer. The nanoparticles are nonporous. Examples of nonporous nanoparticles include silicas used are Snowtex O and Snowtex C made at Nissan Chemical Company in Houston Tex.

In another embodiment, the thioether detection mechanism is coated onto a cellulose substrate which is then inserted into a straw. Yet in another embodiment, the thioether detection mechanism is again coated onto a cellulose substrate, but placed over one end of a straw.

The capture mechanism may include a simple supporting member, such as a transparent or semi-transparent tube (C) or straw (C) containing the thioether detection mechanism. The thioether detection mechanism may be in the form of a powder, in solution, or may be coated onto a substrate, such as cellulose tissue or paper, cotton, silk, a non-woven fabric substrate, a woven substrate, glass fiber, rayon, and so forth. The detection mechanism may also be printed onto the substrate, for example by using an inkjet printer. Or the thioether detection mechanism can be dipped and squeezed, slot coated, applied flexographically, sprayed or gravured.

The straw (C) or tube (C) may be flattened to allow for easier storage, while still permitting the user to blow through the passage in the tube or straw.

In another embodiment, when the user blows into the tube, their breath will pass through the tube and over or through the substrate, thus causing the thioether detection mechanism to discharge minimally or completely (E).

The detection of malaria in a user disclosed herein may be done visually or electronically. The straw or tube may be connected to the inlet of a breath collection balloon (F) such as the type sold commercially by Kimberly-Clark/Ballard Medical Devices of Draper, Utah for use in existing H. pylori detection kits. Such a test balloon has a volume of about 0.5 to 2 liters. The use of such a balloon helps ensure that the correct amount of breath is sampled once the balloon was fully inflated.

Alternatively, the use of a simple optionally detachable bag (F) connected to the exit end of a straw or tube to visually ensure the user blows a lung full of air through the tube (C) or straw (C) may be used. The bag may be made of any material that is compatible with the capture mechanism.

According to another embodiment, the capture mechanism devices described above were made smaller in size so that several of the devices could be easily packaged together in a pocket-sized container. For example, a straw (G) or (I) was cut about 4 cm in length, and a dye-treated substrate (J) or (H) was either inserted into the straw or was placed over one end of the straw. The straw was then flattened by laminating it in a standard business card heated laminator so that air was still able to pass through the tube of the straw.

Additionally a further embodiment depicts a thioether where it can be detected, monitored, or similarly used by any technique known in the art. Examples include but are not limited to, one or more of gas chromatography mass spectrometry, liquid chromatography mass spectrometry, biosensor, an antibody-based detection system, colorimetric assays, near-infrared, selected ion flow tube mass spectrometry and proton transfer reaction mass spectrometry.

A simple LED light measuring system (used in digital pregnancy tests (FIG. 2)) could be used also as a thioether detection mechanism in the tube or straw to measure the discharge of a dye color. The determination as to whether the color dye is discharged in a simple LED light measuring system can be shown on a screen wherein the screen would display either a yes indicating a discharge of color dye or no to indicate no discharge of the color dye.

Alternatively, a screen depicting a strength bar light indicator on the LED light measuring system could be used as well as a thioether detection mechanism. Or optionally a device for measuring blood/alcohol content may also be used in a similar fashion as a thioether detection mechanism.

Further, an electronic-nose (e-nose) sensor or a similar device thereof that is sensitive to thioethers could also be used as a thioether detection mechanism. The e-nose device comprises of single or multiple sensors that operate at various sensitivity to accurately detect the amount of thioether. The sensitivity of the device could be adjusted by changing the power and/or sampling time and/or changing both simultaneously. The response of the sensor (s) are compared with the pre-calibrated response pattern stored in the memory.

The benefits the invention disclosed herein make it unique. It is a sensitive test for detecting the presence of a thioether in a user's breath. Most importantly, the invention can offer a much earlier detection of malaria (at least 2 days) versus standard blood analysis.

Accordingly a further embodiment of the present invention is for a method or kit for detecting the presence of a thioether in a user wherein the capture mechanism and thioether detection mechanism steps can be completed separately or done at the same time.

Overall, it is important to note that the thioethers produced or found in the user's breath can be a result of a medical condition.

The invention will now be described in more detail by way of the following non-limiting examples.

### Example 1

A 20 ml solution of Michler's Hydrol (4, 4′-Bis-(dimethylamino)benzhydrol, Sigma-Aldrich Chemical Company, Milwaukee Wis.) was dissolved in acetonitrile to make a 1 mg/ml concentration. This solution was applied to a KimWipe® (Kimberly-Clark Corporation, Dallas Tex.) via a dipping followed by hanging in the fume hood to dry for 10 minutes. The KimWipe® had previously been dipped into a solution of Snowtex-O (Nissan Chemical Company, Houston Tex.) and allowed to air dry. The coating of Michler's Hydrol turned a deep blue in color. The coated tissue was cut into 1 cm×2 cm samples.

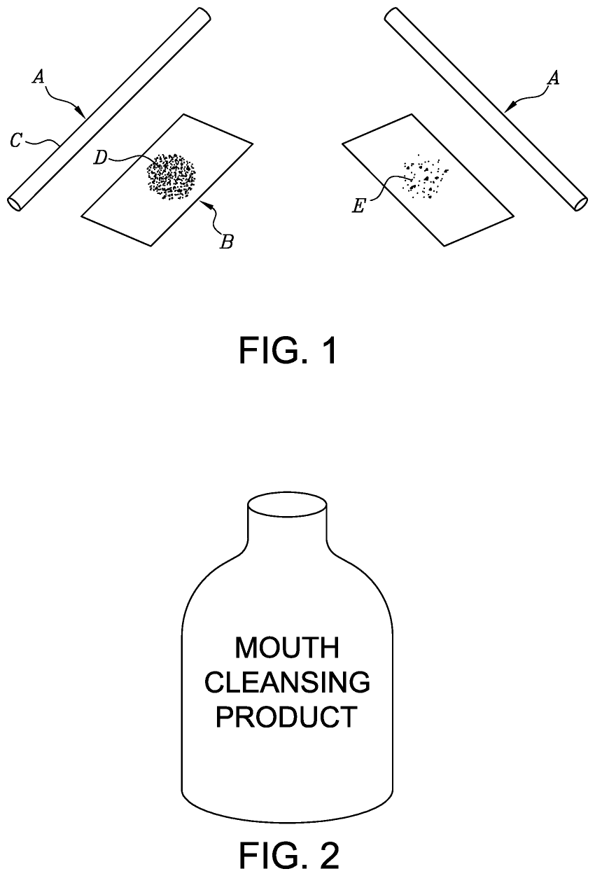

1 Gallon (3.78 litre) air samples containing 20 ppb, 40 ppb and 60 ppb concentrations of 1-methylthio-1-propene (AK Scientific, Inc., Palo Alto Calif.) were prepared by a series of gas dilutions (see FIG. 1) by syringe transfers.

The 1 cm×2 cm Michler's Hydrol coated tissue sample was then placed into a 1 cm diameter, 0.5 cm internal diameter by 5 cm long Tygon tube. Next 120 ml of the air sample containing 20 parts per billion (ppb) of 1-methylthio-1-propene was then injected through the Tygon tube via a 60 ml syringe (3 applications). The change in the blue color was measured by comparing the control blue tissue sample with that of the thioether exposed sample by measuring the ΔE* value (Spectrophotometer Model CM2600d. Minolta Company Ltd., Japan). This experiment was repeated for duplicate analysis. Then this procedure was repeated for the 40 ppb and 60 ppb of 1-methylthio-1-propene containing air samples.

The target would be for the user to blow a lung full of air through the tube (average adult male lung capacity is 5.8 liters and female is 4.2 liters). A simple visual queue for the field medical person would be to a plastic bag at the exit end of the tube that would fill up to show that the subject was indeed exhaling a full lung full.

### Example 2

3 ml solution of 0.1 mg/ml of Michler's Hydrol in Snowtex-O was made and its absorption at 605 nm was measured as A=0.86 in a 1 cm cuvette cell. To this solution was added 25 μL solution of 40 ppb of 1-methylthio-1-propene in water. The absorption dropped to A=0.66. This experiment further shows the sensitivity of Michler's Hydrol to low concentration of thioethers in solution, potentially showing the possibility of using LED detectors as a digital method to measure whether subjects have malaria.

### Example 3

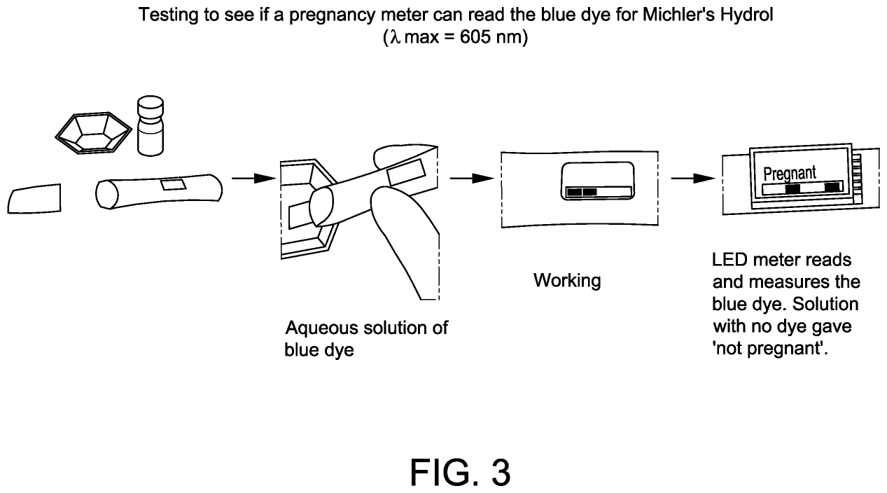

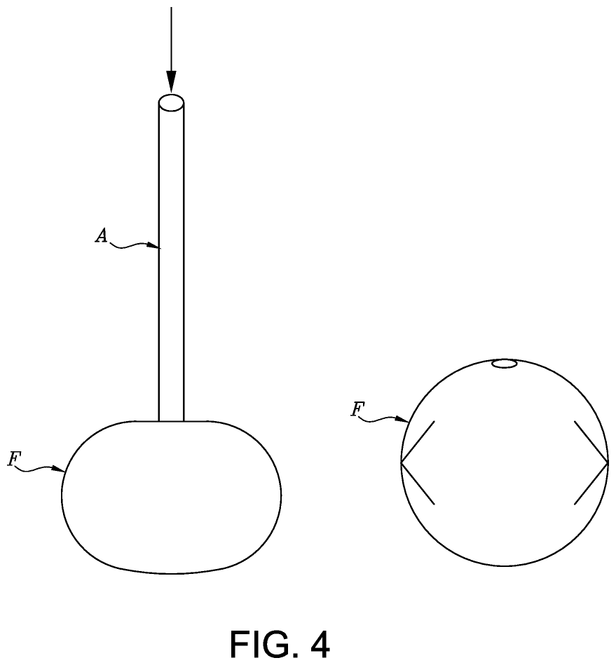

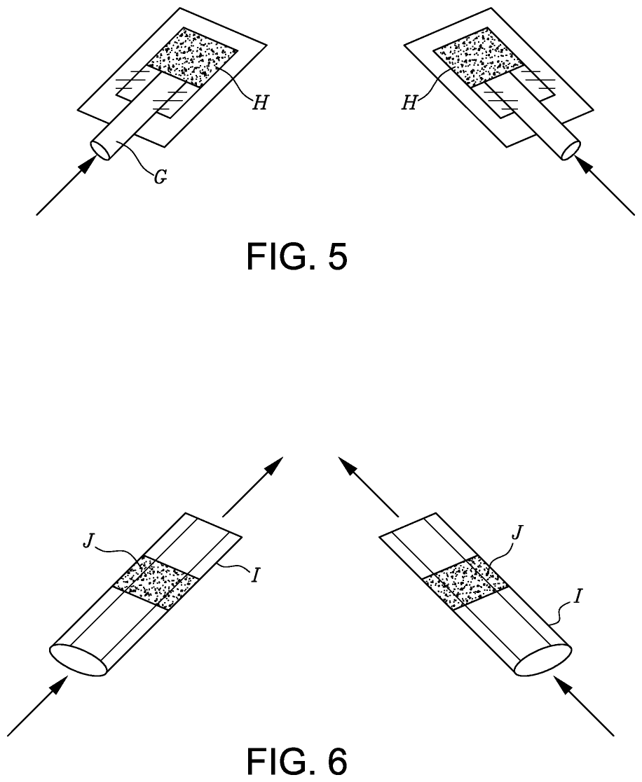

A digital pregnancy tester substrate (Clearblue, SPD Swiss Precision Diagnostics GmbH, distributed by Proctor & Gamble, Cincinnati Ohio) (FIG. 2) was dipped into 0.1 mg/ml Michler's Hydrol in Snowtex-O solution for 20 seconds, as suggested in the instructions and laid horizontal on a bench. After 5 seconds the digital display showed ‘pregnant’ message (see FIG. 3). Repeating this experiment with a new tester and just a Snowtex-O solution gave a ‘Not pregnant’ message. These experiments show that the LED sensor can measure the blue color of the dye and therefore could be modified to measure a decrease in the blue color.

### Example 4

The following gives an example of a method that is used to assess if the subject has a malaria infection:
1. User washes their mouth with mouth wash or other water-based solution for 10 seconds;
2. User exhales a full lung full of breath onto a substrate containing the indicator;
3. The indicator is allowed to react for 20 seconds;
4. The substrate/indicator is a) visually compared to a standard color chart;
5. or a device is activated and the substrate/indicator is b) loaded into a LED device and the message or light displayed to signal no or yes infection or c) the discharge is measured and reading noted and compared to a chart; and
6. The test is optionally repeated as needed for verification.

### Example 5

An embodiment of a capture mechanism (A) for detecting thioethers in the user's breath was designed using a sensitive dye coat on a cellulose substrate (B). Accordingly, 1 mg/ml stock solution of Michler's Hydrol-blue color dye was applied onto a SCOTT® paper towel from Scott Paper of Mississauga, ON, Canada, that had been previously coated with a 1 weight percent (dry) solution of SNOWTEX-O® nanoparticles and allowed to air dry. The dye-coated paper towel was then cut into 2 cm×4 cm strips (H or J) which were rolled up and each strip (H or J) was inserted into a clear plastic drinking straw (G or I) from Glad Products Company of Oakland, Calif.

The devices were tested by injecting known concentrations of thioethers into the straws to determine their sensitivity to sulfur odors. A discharge of the Michler's Hydrol was noticed and was clearly visible in the presence of sulfur odors.

A bag and balloon were attached on one end of the tube to visually check (provider and/or user) that the user exhaled one lung full.

Many modifications and variations of the present disclosure can be made without departing from the spirit and scope thereof. Therefore, the exemplary embodiments described above and below should not be used to limit the scope of the invention.

Additional embodiments specifically disclosed in the invention are as follows:

A method for detecting for the presence of a thioether in a user, the method comprising:

instructing a user to use a mouth cleansing product;

causing the user to blow or breathe onto or into a capture mechanism;

providing a thioether detection mechanism;

analyzing the user's breath with the thioether detection mechanism to determine if the thioether detection mechanism has detected thioethers.

A method for detecting for the presence of a thioether in a user, wherein the method consists of the following steps:

instructing a user to use a mouth cleansing product; then

causing the user to blow or breathe onto or into a capture mechanism; next

providing a thioether detection mechanism; then

analyzing the user's breath with the thioether detection mechanism to determine if the thioether detection mechanism has detected thioethers.

The method according to any of the preceding embodiments, wherein the capture mechanism is a substrate, straw, tube, strip or similar device thereof and wherein the capture mechanism can include the thioether detection mechanism.

The method according to any of the preceding embodiments, wherein the capture mechanism can alternatively include a bag, vial or balloon or later transferred to a thioether detection mechanism such as a gas chromatography mass spectrometry, liquid chromatography mass spectrometry, electronic nose device, biosensor, an or antibody-based detection system.

The method according to any of the preceding embodiments, wherein the thioether detection mechanism consists of one or more of the following: gas chromatography mass spectrometry, liquid chromatography mass spectrometry, electronic nose device, biosensor, an antibody-based detection system, colorimetric assays, near-infrared, selected ion flow tube mass spectrometry and proton transfer reaction mass spectrometry.

A method for detecting for the presence of a thioether in a user, the method comprising: instructing a user to use a mouth cleansing product;

causing the user to blow or breathe onto or into a capture mechanism after the user has used the mouth cleansing product, the capture mechanism comprising a substrate on which a thioether detection composition is disposed, wherein the substrate is located within a carrier portion of a capture detection mechanism through which the breath of a user passes; and

observing whether the substrate discharges the thioether detection mechanism wherein said method is optionally repeated.

The method according to the embodiment for detecting for the presence of a thioether in a user, wherein the thioether detection mechanism is Michler's Hydrol or a similar compound thereof.

The method according to any of the preceding embodiments, wherein the method is used to detect malaria.

A method for detecting for the presence of a thioether in a user, the method comprising:

instructing a user to use a mouth cleansing product;

causing the user to blow or breathe onto or into a capture mechanism after the user has used the mouth cleansing product, the capture mechanism comprising a substrate on which a thioether detection mechanism is disposed, wherein the substrate is located within a carrier portion a breath testing device through which the breath of a user passes; and

observing whether the substrate discharges the thioether detection mechanism wherein said method is optionally repeated.

The method according to any of the preceding embodiments, wherein the method is used to detect malaria.

The method according to any of the preceding embodiments, wherein the mouth cleansing product is Listerine®, Crest®, ACT®, Biotene®, Rembrandt®, Jason®, BreathRX®, CloSYS® or any other similar commercially available mouth rinse, teeth brushing with any commercially available toothpaste, water, baking soda solution, or alcohol solution.

The method according to any of the preceding embodiments, wherein the thioether detection mechanism is 4,4′-bis(dimethylamino)-benzhydrol (Michler's Hydrol).

The method according to any of the preceding embodiments, wherein Michler's Hydrol is a blue color.

The method according to any of the preceding embodiments, wherein the amount of the thioether detection mechanism on the substrate is in the range of 0.01 mg to 10 mg per cm2 of substrate.

The method according to any of the preceding embodiments, wherein the user keeps the mouth cleansing rinse in their mouth for at least 10 seconds.

The method according to any of the preceding embodiments, wherein the carrier portion is open at least on one end.

The method according to any of the preceding embodiments, wherein the carrier portion is a cylindrical, triangular, square, or almond-shaped structure.

The method according to any of the preceding embodiments, wherein the carrier portion is substantially flattened.

The method according to any of the preceding embodiments, wherein the substrate covers the end of a carrier portion.

The method according to any of the preceding embodiments, wherein the substrate comprises at least one of the following: a fibrous material; a non-woven polyproplylene/polyethylene; glass fiber; cotton; silk; or rayon.

The method according to any of the preceding embodiments, wherein the fibrous material contains cellulosic fibers.

A kit for detecting for the presence of a thioether in a user, the kit comprising:

a mouth cleansing product; a capture mechanism; and a thioether detection mechanism

wherein the kit is operative to receive a user's breath.

A kit for detecting for the presence of a thioether in a user, the kit comprising:

a mouth cleansing product; a capture mechanism; and Michler's Hydrol

wherein the kit is operative to receive a user's breath.

The kit for the preceding embodiment, wherein the mouth cleansing rinse is Listerine®, Crest®, ACT®, Biotene®, Rembrandt®, Jason®, BreathRX®, CloSYS® or any other similar commercially available mouth rinse, teeth brushing with any commercially available toothpaste, water, baking soda solution, or alcohol solution.

The kit for any of the preceding embodiments, wherein the capture mechanism is a substrate, strip, straw, tube or any similar device thereof.

The kit for any of the preceding embodiments, wherein the thioether detection composition is Michler's Hydrol.

The kit of an embodiment for detecting the presence of a thioether in a user as described above, wherein the capture mechanism is a straw, tube or an electronic nose or a similar sensor thereof sensitive to thioethers and wherein the capture mechanism can include the thioether detection mechanism.

The kit of an embodiment for detecting the presence of a thioether in a user as described above, wherein the capture mechanism is a straw, tube or an electronic nose or a similar sensor thereof sensitive to thioethers and wherein the capture mechanism can be attached to a balloon, bag, vial or a similar structure thereof.

The kit according to any of the preceding embodiments, wherein the kit is used to detect malaria.

The kit according to any of the preceding embodiments, wherein the thioether detection mechanism is Michler's Hydrol.

The kit according to any of the preceding embodiments, wherein the kit contains 0.01 mg to 10 mg per cm2 of substrate amount of Michler's Hydrol.

The kit according to any of the preceding embodiments, wherein the kit includes instructions instructing the user to keep the mouth cleansing rinse or product in their mouth for at least 1 second, 5 seconds, 10 seconds, 15 seconds, 20 seconds, or more than 20 seconds.

The kit according to any of the preceding embodiments, wherein thioether detection mechanism is contained on a substrate located within the capture mechanism.

When introducing elements of the present disclosure or the preferred embodiment(s) thereof, the articles ‘a’, ‘an’, ‘the’ and ‘said’ are intended to mean that there are one or more of the elements.

The terms ‘comprising’, ‘including’ and ‘having’ are intended to be inclusive and mean that there may be additional elements other than the listed elements.

While the invention has been described in detail with respect to specific embodiments thereof, it will be apparent to those skilled in the art that various alterations, modifications, and other changes may be made to the invention without departing for the spirit and scope of the present invention. It is therefore intended that the claims cover or encompass all such modifications, alterations and/or changes.

What is claimed is:

1. A method for detecting for the presence of a thioether in a user, the method comprising:
instructing a user to use a mouth cleansing product;
causing the user to blow or breathe onto or into a capture mechanism;
providing a thioether detection mechanism;
analyzing the user's breath with the thioether detection mechanism to determine if the thioether detection mechanism has detected thioethers.

2. The method according to claim 1, wherein the capture mechanism is a substrate, straw, tube, strip or similar device thereof and wherein the capture mechanism can include the thioether detection mechanism.

3. The method according to claim 1, wherein the thioether detection mechanism is 4,4′-bis(dimethylamino)-benzhydrol (Michler's Hydrol).

4. The method according to claim 1, wherein the thioether detection mechanism consists of one or more of the following: gas chromatography mass spectrometry, liquid chromatography mass spectrometry, electronic nose device, biosensor, an antibody-based detection system, colorimetric assays, near-infrared, selected ion flow tube mass spectrometry and proton transfer reaction mass spectrometry.

5. The method according to claim 1, wherein the method is used to detect malaria.

6. A method for detecting for the presence of a thioether in a user, the method comprising:
instructing a user to use a mouth cleansing product;
causing the user to blow or breathe onto or into a capture mechanism, the capture mechanism comprising a substrate on which a thioether detection mechanism is disposed, wherein the substrate is located within a carrier portion of a capture detection mechanism through which the breath of a user passes; and
observing whether the substrate discharges the thioether detection mechanism wherein said method is optionally repeated.

7. The method according to claim 6, wherein the method is used to detect malaria.

8. The method according to claim 6, wherein the thioether detection mechanism is 4,4′-bis(dimethylamino)-benzhydrol (Michler's Hydrol).

9. The method according to claim 6, wherein the amount of the thioether detection mechanism on the substrate is in the range of 0.01 mg to 10 mg per cm2 of substrate.

10. The method of claim 6, wherein the user keeps the mouth cleansing rinse in their mouth for at least 10 seconds.

11. The method according to claim 6, wherein the carrier portion is open at least on one end.

12. The method according to claim 6, wherein the carrier portion is a cylindrical, triangular, square, or almond-shaped structure.

13. The method according to claim 6, wherein the carrier portion is substantially flattened.

14. The method according to claim 6, wherein the substrate covers the end of a carrier portion.

15. The method of claim 6, wherein the substrate comprises at least one of the following: a fibrous material; a non-woven polyproplylene/polyethylene; glass fiber; cotton; silk; or rayon.

16. The method according to claim 15, wherein the fibrous material contains cellulosic fibers.

17. A kit for detecting for the presence of a thioether in a user, the kit comprising:
a mouth cleansing product; a capture mechanism; and a thioether detection mechanism wherein the kit is operative to receive a user's breath.

18. The kit according to claim 17, wherein the thioether detection mechanism consists of one or more of the following: gas chromatography mass spectrometry, liquid chromatography mass spectrometry, electronic nose device, biosensor, an antibody-based detection system, colorimetric assays, near-infrared, selected ion flow tube mass spectrometry and proton transfer reaction mass spectrometry.

19. The kit according to claim 17, wherein the thioether detection mechanism is Michler's Hydrol.

20. The kit according to claim 17, wherein the kit is used to detect malaria.
